(19) United States
(12) Patent Application Publication
(10) Pub. No.: US 2003/0228619 A1
(43) Pub. Date: Dec. 11, 2003

(54) Peptide nucleic acids as tags in encoded libraries

(75) Inventor: Michael C. Needels, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XenoPort, Inc., Santa Clara, CA

(21) Appl. No.: 10/444,455

(22) Filed: May 22, 2003

Related U.S. Application Data
(60) Provisional application No. 60/387,780, filed on Jun. 10, 2002.

(51) Int. Cl. C12Q 1/68; C07K 14/00

(52) U.S. Cl. 435/6; 530/350

(57) ABSTRACT

Methods of screening a library of compounds are disclosed herein. The methods include providing a library of differing complexes. Each complex includes a compound and a peptide nucleic acid tag encoding one or more synthesis steps by which the compound was made, the compounds and the tags encoding them differing between the different complexes. Usually, the complex is cleavably bound to a support. One preferred support is polyethylene glycol (PEG), and optionally the PEG support is attached to a dendrimer. The library of differing complexes is then contacted with a molecular target (e.g., a receptor or enzyme). A complex(es) that bind to the target is isolated. The isolated complex(es) is contacted with a nucleic acid that hybridizes to the tag of the complex. The tag is decoded from the identity of the nucleic acid that hybridizes to the tag, thereby identifying the compound which binds to the target.

### CROSS-REFERENCE TO RELATED APPLICATION

The present application is a nonprovisional of U.S. Ser. No. 60/387,780, filed Jun. 10, 2002 which is incorporated by reference in its entirety for all purposes.

### BACKGROUND

Methods for synthesizing and screening encoded synthetic libraries (ESL) have previously been described (Affymax, WO 95/12608, Affymax, WO 93/06121, Columbia University, WO 94/08051, Pharmacopeia, WO 95/35503 and Scripps, WO 95/30642). In these methods, the different compounds in the library are usually synthesized attached to separate supports (e.g., beads) by stepwise addition of the various components of the compounds in several rounds of coupling. A round of coupling can be performed by apportioning the supports between different reaction vessels and adding a different component to the supports in the different reaction vessels. The particular component added in a reaction vessel can be recorded by the addition of a tag component to the support at a second site. The types of tags used to date include nucleic acids, which can be decoded by PCR and sequencing, and so-called hard tags, which can be decoded by mass spectrometry. After each round of synthesis, supports from the same reaction vessel can be apportioned between different reaction vessels and/or pooled with supports from another reaction vessel in the next round of synthesis. In any, and usually in all rounds of synthesis, the component added to the support can be recorded by addition of a further tag component at a second site of the support. After several rounds of synthesis, a large library of different compounds is produced in which the identities of compounds are encoded in tags attached to the respective supports bearing the compounds. The library can be screened for binding to a target. Optionally, compounds linked to tags can be detached from supports before screening. Compounds having a specific affinity for the target are isolated, and the identity of such compounds can be determined by decoding the tags.

Brenner and Lemer in U.S. Pat. No. 5,573,905 also discuss an encoding technology using single-stranded DNA tags. In this patent, they discuss the possibility of using the DNA tag as not only an encoding molecule, but also as a means to prepare a derivative of the primary library which is enriched in compounds which were found to be hits during a screen. Thus, hit molecules, tethered to their single stranded DNA tags, are contacted with the entirety of the primary library. This enriched, secondary library can then be re-screened to provide more sensitive detection of very rare hits. This method of library enrichment has not been widely used, primarily because of the fundamental deficiencies of DNA tags: they are not chemically robust to many of the reactions commonly performed in combinatorial synthesis of non-polymeric, heterocyclic compounds.

### BRIEF DESCRIPTION OF THE FIGURES

FIG. 1 is a schematic diagram showing one example of a screening method;

FIG. 2 is a schematic diagram showing an alternate screening method using a dendritic carrier;

FIG. 3 is a schematic diagram showing an alternate screening method using a single stranded plasmid carrier; and

FIG. 4 is a schematic diagram showing a peptide nucleic acid-encoded, biotin-labeled, cell-permeable compound library prepared on a polyethylene glycol support.

### SUMMARY

Methods of screening a library of compounds are disclosed herein. Such methods entail providing a library of complexes wherein a complex comprises a compound, a peptide nucleic acid tag encoding one or more synthetic steps by which the compound was produced, the compounds and the tags encoding them differing between the complexes. In one of the disclosed methods, the library of complexes is contacted with a target. A complex that binds to the target is isolated. The isolated complex is contacted with a nucleic acid that hybridizes to the tag of the complex. The tag is decoded from the identity of the nucleic acid that hybridizes to the tag. In another disclosed method, the library of complexes is contacted with a cell. A complex that permeates into the cell is isolated and contacted with a nucleic acid that hybridizes to the tag of the complex. The tag is decoded from the identity of the nucleic acid that hybridizes to the tag.

### DEFINITIONS

A “peptide nucleic acid” (also abbreviated herein as “PNA”) includes at least two monomer units of nucleobase subunits linked by a peptide bond. The monomers can be the natural bases A, C, G, T or U or synthetic analogs thereof.

A conventional “amino acid”, or “nucleic acid”, is as defined in the World Intellectual Property Organization (WIPO) Handbook on Industrial Property Information and Documentation, Standard ST.25: Standard for the Presentation of Nucleotide and Amino Acid Sequence Listings in Patent Applications (1998), including Tables 1 through 6 in Appendix 2.

An “encoded library” is a library of compounds in which each compound is linked to a tag recording at least one step in synthesis of the compound.

### DETAILED DESCRIPTION

1. General

Disclosed herein are coded synthetic libraries and methods of screening the same that offer a number of advantages over previous methods. In one aspect of the invention, coded synthetic libraries are synthesized in which the tag is a peptide nucleic acid (PNA) rather than a conventional nucleic acid. Peptide nucleic acids offer the same advantages of conventional nucleic acids in terms of coding capacity. That is, the number of coding possibilities increases by a factor of four for each position in the peptide nucleic acid, such that relatively short peptide nucleic acids can encode and distinguish between a vast number of different synthetic schemes. PNAs offer an advantage over conventional nucleic acids in that the former are more resilient to the types of chemical reactions conventionally used in synthesis of compounds. Therefore, use of PNAs as tags is compatible with more synthetic schemes than conventional nucleic acids. An apparent disadvantage of PNA tags relative to conventional nucleic acids is that PNA tags cannot be amplified by PCR nor decoded by conventional sequencing approaches such as use of dideoxynucleotides. However, PNA tags can be decoded indirectly by hybridizing the tags to a conventional nucleic acid, and then decoding the conventional nucleic acid. PNAs hybridize according to conventional Watson-Crick base pairing. Hence, the identity of a hybridizing conventional nucleic acid reveals the identity of the PNA tag to which it is hybridizing as well.

The conventional nucleic acid can be detected by amplification followed by conventional sequencing. In some methods, amplification is by PCR or a similar in vitro amplification method. In other methods, the conventional nucleic acid is a component of a vector, and amplification is performed by transformation into host cells and harvesting the amplified vector. In other methods, the conventional nucleic acid is detected indirectly as a result of further hybridization to a second conventional nucleic acid. The second nucleic acid can be detected by amplification, either in vivo or in vitro, followed by a conventional sequencing method. Determination of the sequence of the second nucleic acid (or part thereof) reveals the sequence of the complementary conventional nucleic acid that hybridizes to the tag, and hence the sequence of the PNA tag. In some methods, the conventional nucleic acid that hybridizes to the tag is provided in immobilized form, for example, immobilized to a bead. Such a bead can bear multiple copies of the conventional nucleic acid and thus can hybridize simultaneously to the PNA tag and to a second conventional nucleic acid forming a bridge between them.

During synthesis of PNA tagged libraries, the nascent PNA tag and compound are linked to one another and usually to a support as well. PNA tag and compound can be independently linked to the support, or through “T” shaped arrangement in which the PNA tag and compound occur at opposite ends of the horizontal bond, and the support occurs at the bottom of the vertical bond. In such an arrangement, the horizontal bond is typically noncleavable and the vertical bond can be cleavable. Cleavage of the vertical bond releases the PNA tag and compound from the support while they remain linked to each other.

In a preferred method, a polyethylene glycol (PEG) support is used. A PEG support is useful since it is soluble during steps of chemical synthesis in most organic solvents (e.g., methylene chloride, CHCl₃, tetrahydrofuran (THF), and dimethyl formamide (DMF), but is transformed into an insoluble matrix during wash steps by exposure to diethyl ether or cold ethanol. The use of linear PEG supports for unencoded combinatorial synthesis has been described in WO96/03418; and in Janda and Han, Methods in Enzymology, v. 267, pps. 234-247 (1996).

In a further preferred method, a linear PEG support is attached to a dendrimer. Combinatorial synthesis and encoding chemistry take place on suitable functionalities of the dendrimer. Alternatively, dendrimers with a branched PEG core can be used. In either case, a differentially soluble dendrimer is created. The use of a dendrimer provides a higher loading of compound-PNA conjugate for every differentially soluble molecule.

2. Peptide Nucleic Acids

Peptide nucleic acids (PNAs) are analogs of nucleic acids in which the ribose-phosphate backbone has been replaced with a backbone that is held together by amide bonds as described in U.S. Pat. No. 5,539,082 to Nielsen et al. PNAs hybridize to complementary DNA, RNA, or peptide nucleic acid (PNA) sequences. The hybridization binding affinity of a PNA to a complementary sequence is generally greater than that of nucleic acid to nucleic acid hybrids (Good and Nielsen (1997) Antisense and Nuc Acid Drug Dev (7)431-7). PNAs can be synthesized as described by Egholm et al., J. Am. Chem. Soc. 1992, 114, 9677-9678; Buchardt et al., WO 92/20702) using the acid-labile tert-butyloxycarbonyl (Boc) protecting group, which is eliminated with medium-strength acids such as, for example, trifluoroacetic acid, for temporarily protecting the amino group of the monomer. Synthesis of oligomers then follows the customary peptide synthesis method as described, for example, by B. Merrifield in, J. Am. Chem. Soc., 1963, 85, 2149. A strong acid, usually liquid hydrogen fluoride, is used in this case to cleave the PNA oligomer from the support. See G. Jung and A. Beck-Sickinger, Angew. Chem. 104 (1992) 375-391. PNAs can also be synthesized using a base-labile, temporary amino protecting group for constructing the PNA oligomers and which permits cleavage of the oligomer from the support using weak or medium-strength acids as described by U.S. Pat. No. 6,316,595.

3. Synthesis of Encoded Libraries

Preparation of encoded libraries is described in a variety of publications including Needels, et al. Proc. Natl. Acad. Sci. USA 1993, 90, 10700; Ni, et al. J. Med. Chem. 1996, 39, 1601, WO 95/12608, WO 93/06121, WO 94/08051, WO 95/35503 and WO 95/30642 (each of which is incorporated by reference in its entirety for all purposes). Methods for synthesizing encoded libraries typically involve a random combinatorial approach and the chemical and/or enzymatic assembly of component units. For example, the method typically includes steps of: (a) apportioning a plurality of supports (preferably differentially soluble supports, e.g. PEG) among a plurality of reaction vessels; (b) coupling to the supports in each reaction vessel a first component of compounds to be synthesized, and a first component of a PNA tag; the first components of the compound and the tag differing between different reaction vessels; (c) pooling the supports; (d) apportioning the supports among a plurality of reaction vessels; (e) coupling to the first component of the compounds a second component, and coupling to either the support or to the first component of the PNA tag a second component of the PNA tag using different second components of the compound and PNA in each different reaction vessel; and optionally repeating the coupling and apportioning steps with different compound and tag components one to twenty or more times. The set of components for compound synthesis can be expanded or contracted from step to step; or the set could be changed completely for the next step (e.g., amino acids in one step, nucleosides in another step, carbohydrates in another step). A component for peptide synthesis, for example, can include single amino acids or larger peptide units, or both. Likewise, components for PNA synthesis can include single nucleotides or PNA oligomers in which the monomers are joined by PNA bonds.

PNA tags are more robust to a broader range of chemistries than are DNA tags. Therefore, PNA tags are useful for a wide variety of combinatorial chemistries that produce non-linear, non-polymeric, heterocyclic molecules.

The relative time at which the components of a compounds and encoding tags are attached to a support is not critical. For example, a component of an encoding tag can be attached immediately before, during, or after a round of addition of a component of compounds, so long as such timing is compatible with modes of attachment, and the chemistries involved in preparing the compound and tag. The necessary encoding of the synthesis steps can be achieved using a single or multiple tag components. The size of libraries generated by such methods can vary from 2 different compounds to 10², 10⁴, 10⁶ or 10⁸ compounds.

In some instances, the compound-PNA conjugate remains permanently attached to the differentially soluble support during screening using a suitable linker. In a preferred method, compound-PNA conjugates are cleaved from supports before screening by employing a cleavable linker. The linkers typically are bifunctional (i.e., the linker contains a functional group at each end that is reactive with groups located on the element to which the linker is to be attached); the functional groups at each end can be the same or different. Examples of suitable linkers include straight or branched-chain carbon linkers, heterocyclic linkers and peptide linkers. Exemplary linkers that can be employed in the present invention are available from Pierce Chemical Company in Rockford, Ill. and are described in European Patent Application 188,256; U.S. Pat. Nos. 4,671,958; 4,659,839; 4,414,148; 4,669,784; 4,680,338, 4,569,789 and 4,589,071; and Eggenweiler, H. M, Pharmaceutical agent Discovery Today 1998, 3, 552.

The choice of linker depends on whether the linker is intended to remain permanently in place or is intended to be cleaved before or during screening. For cleavage prior to use, NVOC (6-nitroveratryloxycarbonyl) linkers and other NVOC-related linkers are examples of suitable photochemical linkers (see, e.g., WO 90/15070 and WO 92/10092), as are nucleic acids with one or more restriction sites, or peptides with protease cleavage sites (see, e.g., U.S. Pat. No. 5,382,513). Commercially available acid cleavable linkers (e.g., Rink and Wang type linkers), photolinkers and linkers cleavable with palladium chemistry (e.g., aloc linkers) may also be used. One commercial source of such linkers is Calbiochem-Novabiochem of Laufelfingen, Switzerland. Specific examples of such commercially available linkers include: 4-[4-(1-(Fmocamino)ethyl)-2-methoxy-5-nitrophenoxy)butanoic acid; p-[(R,S)-a-[1-(9H-Fluoren-9-yl)-methoxyformamido]-2,4-dimethoxybenzyl]-phenoxyacetic acid; 4-(4-formyl-3-methoxy-phenoxy)-butyric acid; 4-Hydroxymethylbenzoic acid; 4-Hydroxymethylphenoxyacetic acid; 4-(4-Hydroxymethyl-3-methoxyphenoxy)-butyric acid; 4-[4-(1-Hydroxyethyl)-2-methoxy-5-nitrophenoxy)-butanoic acid; 4-[4-Hydroxymethyl-2-methoxy-5-nitrophenoxy)-butanoic acid; N-Fmoc-N-methoxy-3-aminopropionic acid; p[(R,S)-a-[1-(9H-Fluoren-9-yl)-methoxyformamido]-2,4-dimethoxybenzyl]-phenoxyacetic acid; 3-Hydroxy-xanthen-9-one; and N-Fmoc-N-methoxy-3-aminopropionic acid. For cleavage during use, one selects a linker that is spontaneously cleaved under the conditions of the relevant assay (usually a physiological buffer). Compounds synthesizable include, for example, peptides, oligonucleotides, oligo N-substituted glycines, and polycarbamates. Other compounds include polymers formed from one or more of the following monomer types: amino acids, carbamates, sulfones, sulfoxides, nucleosides, carbohydrates, ureas, phosphonates, lipids, esters. Other compounds that can be synthesized in a component-by-component fashion can also be screened including benzodiazepines, hydantoins, and peptidylphosphonates.

The encoding tags encode one or more reaction steps taken in synthesizing the test compound. For those test compounds wherein the synthesis yields a single product in high yield (e.g., peptide and oligonucleotide synthesis), the tag explicitly specifies one, and usually all, of the components of the test compound and its structure. In some situations, for example, when only a small number of monomer units of an oligomer are varied, it is not necessary to identify all the monomers utilized in the synthesis, but only those monomers which vary among the oligomers. For other syntheses that give variable yields and frequently multiple products (such as regio- and stereoisomeric structures), a mixture of compounds is sometimes obtained on each bead. In such instances, the encoding tag can not uniquely specify the chemical structure of the synthesized test compound. Instead, the encoding tag encodes the synthetic protocol (e.g., reagents and reaction conditions) by which a test compound in a library was prepared.

### EXAMPLES

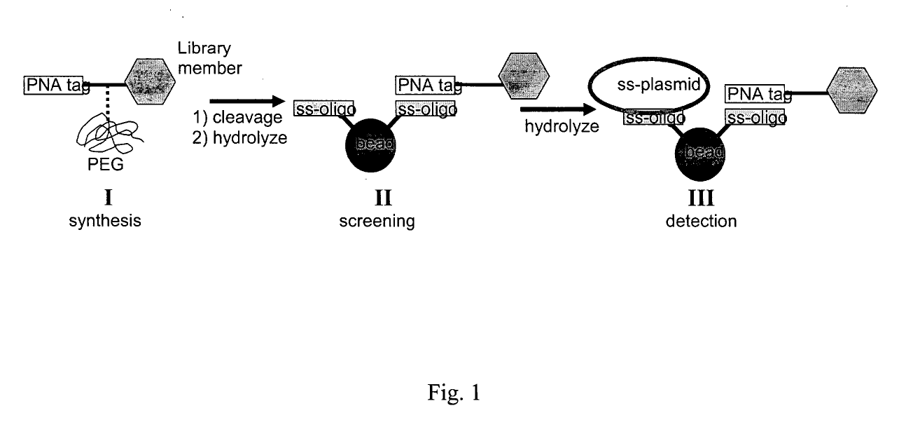

An exemplary embodiment of the invention is represented schematically in FIG. 1, and involves the following steps:

(1) Library Synthesis. The library is synthesized using an encoded combinatorial synthesis on a differentially soluble PEG support (see WO 96/03418). Each member of the library is directly attached to a linker molecule. This linker is directly attached to a PNA tag as well as the PEG support. The connection between the linker and the PEG support is usually cleavable (e.g. acid or photolysis). However the linkage between the compound and the PNA tag is not usually cleavable. The PNA tag is synthesized in parallel with the combinatorial synthesis of the compound. Different PNA nucleobases are used to encode the identity of the monomer used in the combinatorial synthesis. A PNA tag is more robust than a DNA tag towards reactions performed in a combinatorial synthesis.

(2) Library Screening. Once the library has been synthesized, each compound, along with its attached PNA tag, is cleaved from the PEG support. The entire library is cleaved as a mixture, so a mixture of compound-PNA tag molecules is obtained. Separately, a library of single stranded DNA molecules is prepared on beads. The sequences represented in this library are chosen such that each member has a complementary partner present in the compound-PNA library. The entire ssDNA-bead library is mixed with the compound-PNA library under hybridization conditions. In this way, each bead bearing many copies of a single DNA sequence will hybridize to a number of compound-PNA conjugates all having the same complementary sequence (see structure II in FIG. 1). The library, now displayed on beads, is screened in a manner analogous to “panning” of phage display libraries. The library as a slurry is placed in contact with a molecular target (e.g., a receptor or enzyme) which is immobilized on a solid surface (e.g., beads, wells, or plates). Compounds with affinity for the target adhere to the solid surface. Compounds which do not have affinity for the target are washed away along with their accompanying beads.

(3) Hit Identification. Hits are identified using a second nucleic acid that hybridizes to the oligonucleotides attached to beads. Specifically, a library of ss-plasmid DNA is prepared in which each of the coding sequences used in the original compound-PNA library is present. This library is contacted with the pool of hits isolated from the screening operation described above. Those plasmids whose complements are present in the hit pool are retained on the support used for screening. Structures such as III shown in FIG. 1 are formed. This plasmid DNA can then be amplified either by PCR or transformation into E. coli (after conversion to ds-plasmid). DNA sequencing reveals the structure of the active compound.

Alternatively, rather than directly sequencing the DNA, the ability of the plasmid/ss-DNA-bead/compound-PNA construct to self-assemble can be employed to provide enrichment of the initial hit pool. After amplification of the captured plasmid, the plasmid can be immobilized (e.g. standard blotting techniques or use of a biotinylated ss-oligo) on a solid support. The immobilized plasmid is then treated first with the ssDNA-bead library, and then with the compound-PNA library. The ssDNA-bead/compound-PNA assembly can then be liberated from the support by suitable denaturation conditions (heat or chaotropic reagents). The greater stability of a PNA:DNA vs. a DNA:DNA duplex can be employed to liberate the assembly intact. This now enriched library is used for a subsequent round of screening.

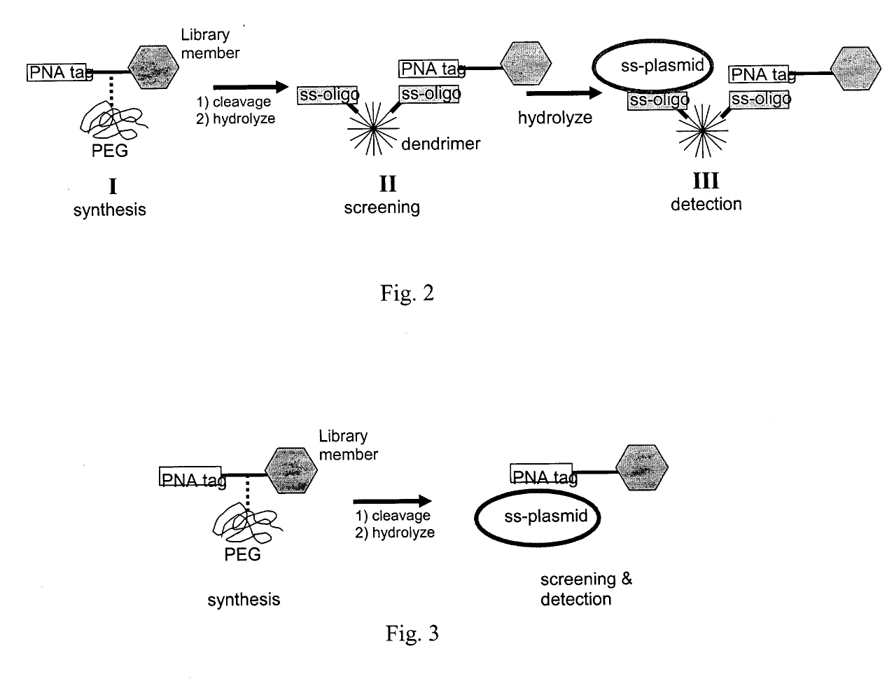

(4) Types of Carriers. In the example provided above, beads were used as the carrier. Other carriers that allow for the multivalent display of ssDNA can also be used. If larger beads are used (i.e. 1-10 microns diameter), a fluorescence activated cell sorting (FACS)-based screening methodology can be used as an alternative to panning. To employ panning, beads having a diameter <1 micron are preferred. Alternatively, dendrimers can be used as the carrier, in place of the bead (FIG. 2) followed by panning. In some methods, the ss-plasmid itself is used as the carrier (FIG. 3). In this case, multiple copies of a sequence complementary to the PNA encoding sequence are present on each plasmid.

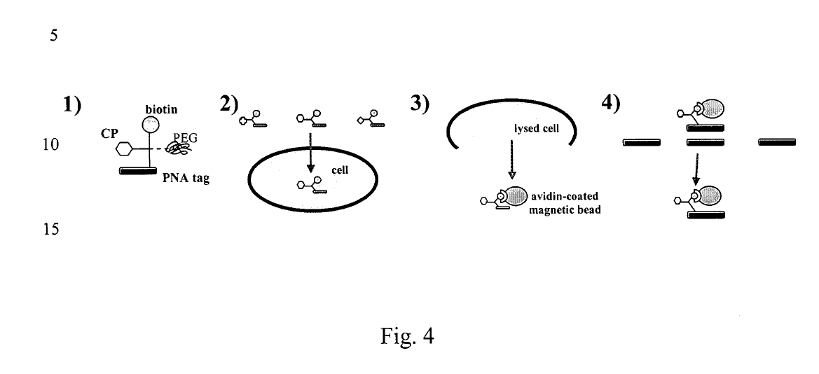

The methods described herein can also be used to identify cell permeable compounds (CPCs) which can be useful for applications such as (i) improving uptake of compounds into enterocyte cells lining the gastrointestinal lumen for improving the bioavailability of orally-administered drugs, and (ii) increasing absorption of drugs or diagnostic agents (e.g., imaging/contrast agents) into specific target cells, for example absorption of an anticancer drug into tumor cells. In both of these applications, the CPC which is identified can be used as a “promoiety” for assisting the transport of an existing compound (i.e., existing drug or diagnostic agent) into a target cell or tissue by linking the CPC to the existing compound to create a conjugate that exhibits improved uptake into the cells compared to the existing compound. The method employs PNA encoding and can be used for modeling CPC-compound (i.e., conjugate) transport of therapeutic and diagnostic agents including low, intermediate and high molecular weight compounds (e.g., diagnostic agents, drugs, peptides, and proteins). A combinatorial library of CPC candidates is prepared on a differentially soluble PEG support. Each combinatorially synthesized compound is attached directly to a unique PNA tag that codes for the building blocks employed in the construction of that compound, and to a biotin molecule. The compound-PNA-biotin conjugates are cleaved from the PEG support and applied to cells in vitro as shown schematically in FIG. 4. In step (1) a member of a PNA-encoded, biotin-labeled CPC library is prepared on a PEG support. In step (2), the library members are cleaved from the PEG support and then contacted with cells. Some of the library members are translocated across the cell membrane, thereby entering the cell. The treated cells are washed to remove library members that have not translocated into the cells. In this way, library members that are unable to enter the cell are washed away. In step (3), the washed cells are lysed, and those library members that have entered the cell are captured and isolated by binding to avidin-coated magnetic beads. In step (4), the bead-bound library members are treated with a library of ss-DNA, where a DNA sequence complementary to each PNA sequence present in the original CPC library is represented. Those ss-DNA molecules having a sequence complementary to one of the PNA tags attached to the magnetic beads are retained. Non-hybridized DNA is washed away, and the retained DNA is amplified and sequenced to elucidate the structure of the CPC.

While the foregoing invention has been described in some detail for purposes of clarity and understanding, it will be clear to one skilled in the art from a reading of this disclosure that various changes in form and detail can be made without departing from the true scope of the invention. The above examples are provided to illustrate the invention, but not to limit its scope; other variants of the invention will be readily apparent to those of ordinary skill in the art and are encompassed by the claim(s) of any patent(s) issued herefrom. The scope of the invention should, therefore, be determined not with reference to the above description, but instead should be determined with reference to the issued claim(s) along with their full scope of equivalents. All publications, references, and patent documents cited in this application are incorporated by reference in their entirety for all purposes to the same extent as if each individual publication or patent document were so individually denoted.

1. A method of screening a library of compounds, comprising
(a) providing a library of complexes wherein a complex comprises a compound, a peptide nucleic acid tag encoding one or more synthetic steps by which the compound was produced, the compounds and the tags encoding them differing between the complexes;
(b) contacting the library of complexes with a target,
(c) isolating a complex that binds to the target;
(d) contacting the complex with a nucleic acid that hybridizes to the tag of the complex; and
(e) decoding the tag from the identity of the nucleic acid that hybridizes to the tag.

2. The method of claim 1, further comprising amplifying the nucleic acid that hybridizes to the tag and sequencing it.

3. The method of claim 2, wherein the nucleic acid is a plasmid that is amplified by transformation in a host.

4. The method of claim 2, wherein the nucleic acid is amplified by PCR.

5. The method of claim 1, further comprising contacting the nucleic acid that hybridizes with the tag with a second nucleic acid which hybridizes to the nucleic acid, and the identity of the nucleic acid is determined from the identity of the second nucleic acid.

6. The method of claim 1, wherein the nucleic acid in step (d) is immobilized to a support.

7. The method of claim 6, wherein multiple copies of the nucleic acid in step (d) are immobilized to the support.

8. The method of claim 7, wherein a first of the copies of the nucleic acid binds to the peptide nucleic acid tag, and a second of the copies of the nucleic acid binds to a second nucleic acid.

9. The method of claim 8, wherein the second nucleic acid is a plasmid.

10. The method of claim 1, wherein the complex further comprises a support attached to the compound and/or the peptide nucleic acid tag.

11. The method of claim 10, wherein the support is polyethylene glycol.

12. A complex comprising a compound linked to a peptide nucleic acid tag that encodes a step of synthesis that produced the compound.

13. A plurality of different complexes, wherein a complex comprises a compound, a peptide nucleic acid tag encoding one or more synthetic steps by which the compound was produced, the compounds and the tags encoding them differing between the complexes.

14. A method of screening a library of compounds, comprising
(a) providing a library of complexes wherein a complex comprises a compound, a peptide nucleic acid tag encoding one or more synthetic steps by which the compound was produced, the compounds and the tags encoding them differing between the complexes;
(b) contacting the library of complexes with a target cell;
(c) isolating a complex that is taken up by the cell;
(d) contacting the complex with a nucleic acid that hybridizes to the tag of the complex; and
(e) decoding the tag from the identity of the nucleic acid that hybridizes to the tag.

15. The method of claim 14, further comprising amplifying the nucleic acid that hybridizes to the tag and sequencing it.

16. The method of claim 15, wherein the nucleic acid is a plasmid that is amplified by transformation in a host.

17. The method of claim 15, wherein the nucleic acid is amplified by PCR.

18. The method of claim 14, further comprising contacting the nucleic acid that hybridizes with the tag with a second nucleic acid which hybridizes to the nucleic acid, and the identity of the nucleic acid is determined from the identity of the second nucleic acid.

19. The method of claim 14, wherein the nucleic acid in step (d) is immobilized to a support.

20. The method of claim 14, wherein multiple copies of the nucleic acid in step (d) are immobilized to the support.

21. The method of claim 20, wherein a first of the copies of the nucleic acid binds to the peptide nucleic acid tag, and a second of the copies of the nucleic acid binds to a second nucleic acid.

22. The method of claim 21, wherein the second nucleic acid is a plasmid.

23. The method of claim 14, wherein the complex further comprises a support attached to the compound and/or the peptide nucleic acid tag.

24. The method of claim 23, wherein the support is polyethylene glycol.

25. A conjugate comprising a compound identified by the screening method of claim 14 covalently linked to a therapeutic agent or diagnostic agent.

26. The conjugate of claim 25, wherein the agent is a therapeutic agent.

27. The conjugate of claim 26, wherein the therapeutic agent comprises a drug.

28. The conjugate of claim 25, wherein the agent is a diagnostic agent.

29. The conjugate of claim 28, wherein the diagnostic agent comprises an image contrast agent.

30. The method of claim 14, wherein the cell comprises an enterocyte.

31. The method of claim 14, wherein the cell comprises a tumor cell.
